(19) United States
(12) Patent Application Publication
(10) Pub. No.: US 2010/0069979 A1
(43) Pub. Date: Mar. 18, 2010

(54) Methods for Determining a Vulnerable Window for the Induction of Fibrillation

(76) Inventor: Michael R. Pittaro, New Canaan, CT (US)

Correspondence Address: MINTZ, LEVIN, COHN, FERRIS, GLOVSKY AND POPEO, P.C, One Financial Center, BOSTON, MA, 02111, US

(21) Appl. No.: 12/210,117

(22) Filed: Sep. 12, 2008

(51) Int. Cl. A61N 1/39

(52) U.S. Cl. 607/5

(57) ABSTRACT

Aspects of the invention include methods for determining a vulnerable window for the induction of fibrillation. The method includes obtaining an intracardiac waveform from a subject's heart; measuring an interval between a plurality of time points on the intracardiac waveform; and evaluating the morphology of the waveform so as to determine the optimal vulnerable window for the induction of fibrillation. Also provided are methods for delivering a stimulus to the heart of a subject during the determined vulnerable window.

### CROSS-REFERENCED TO RELATED APPLICATIONS

This application claims the benefit under 35 U.S.C. Section 119(e) of provisional application U.S. Ser. No. 60/933,679, filed on Sep. 12, 2007 and entitled “METHODS FOR DETERMINING A VULNERABLE WINDOW FOR THE INDUCTION OF FIBRILLATION” which is incorporated herein by reference.

### BACKGROUND OF THE DISCLOSURE

Cardiac arrhythmias such as ventricular fibrillation and/or tachycardia can be life threatening. A common method for treating a heart experiencing ventricular fibrillation and/or tachycardia involves defibrillation of the heart. Several defibrillation devices have been developed. Defibrillation devices, or defibrillators, deliver a therapeutic amount of electrical energy to an affected heart, thereby depolarizing a critical mass of the heart muscle, terminating the arrhythmia, and allowing the re-establishment of normal sinus rhythm. There are several types of defibrillators well known in the art. One such type of defibrillator is an implantable cardiac defibrillator (ICD).

In certain instances, however, it may be desirable to induce fibrillation in a subject. For instance, in certain instances, it is desirable to induce fibrillation for the purpose of upper limit of vulnerability (ULV), or non-ULV, testing of a defibrillator, such as an ICD. For example, a defibrillator may be tested by delivering an electrical current (e.g., a shock) to a subject's heart at a predetermined time during an intracardiac waveform, while the device monitors for the induction of fibrillation. Once the device records the fibrillation, it delivers a defibrillation shock so as to terminate the fibrillation. In this manner, the functioning and/or efficacy of the defibrillation system may be tested. Conversely, with other devices or applications such as with a Taser, it may be desirable to know when a shock may be delivered to the heart so as not to induce fibrillation.

In order to ensure optimal testing conditions and to avoid injury to the heart, it is desirable to determine that vulnerable period of the cardiac cycle wherein cardiac fibrillation may be induced by the delivery of an electrical current. That vulnerable period of the cardiac cycle during which an electrical stimulus can induce fibrillation is commonly known as the vulnerability window. Precise determination of the vulnerability window is useful in identifying the optimal ULV and the defibrillation threshold (DFT) without the application of unnecessary shocks.

Hence, if one merely tries to estimate the ULV, for instance, by delivering one or more induction shocks to the heart so as to scan and search for the vulnerability window, the vulnerability window may be inaccurately determined and may result in a suboptimal result for the patient. Specifically, while the delivery of an electrical current to the heart during a period that is outside of the optimal ULV window may still result in fibrillation, often times such shocks are of sub-ULV values, and therefore may result in an estimate of the DFT that is erroneously low. In this situation, multiple shocks may need to be delivered to successfully defibrillate the patient. Similarly, in non-ULV testing, inaccurate determination of the optimum vulnerability window can lead to difficulty in initiating fibrillation. The application of these additional shocks in ULV and non-ULV testing can lead to prolonged procedural times, increased patient discomfort, and enhanced risk to the subject, including damage to the heart, and death.

Accordingly, there continues to be a need for precisely determining the window of vulnerability for the optimal delivery of a stimuli to the heart so as to induce fibrillation. The present invention meets this and other such needs.

### SUMMARY OF THE DISCLOSURE

Aspects of the disclosure include methods for determining a vulnerable window, for instance, for the induction of fibrillation. The method includes obtaining a waveform, such as an intracardiac waveform, from a subject's heart; evaluating the morphology of the waveform; measuring an interval between a plurality of time points on the waveform, e.g., intracardiac waveform, potentially including the pacing stimulus as a point, so as to determine the vulnerable window for the induction of fibrillation. Also provided are methods for delivering a stimulus to the heart of a subject during the determined vulnerable window to induce fibrillation.

### BRIEF DESCRIPTION OF THE DRAWING

According to common practice, the various features of the drawing may not be drawn to-scale. Rather, the dimensions of the various features may be arbitrarily expanded or reduced for clarity. Included in the drawing is the following figure:

FIG. 1 illustrates an example of an intracardiac waveform in accordance with the disclosure. As is well known to one in the art, an intracardiac waveform may differ from patient to patient, and the following waveform is used for illustrative purposes and is not meant to limit the scope of the invention.

Before the present invention is further described, it is to be understood that this invention is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications may be cited. The disclosures of these publications, patents and published patent applications referenced in this application are hereby incorporated by reference in their entirety into the present disclosure. Citation herein by the Applicant of a publication, patent, or published patent application is not an admission by the Applicant of said publication, patent, or published patent application as prior art.

It must be noted that as used herein and in the appended claims, the singular forms “a”, “and”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like, in connection with the recitation of claim elements, or the use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DETAILED DESCRIPTION

Aspects of the disclosure include methods for determining a vulnerable window, for instance, for the induction of fibrillation. The method includes obtaining a waveform, such as an intracardiac waveform, from a subject's heart; evaluating the morphology of the waveform; and measuring an interval between a plurality of time points on the waveform, potentially including the pacing stimulus, so as to determine a vulnerable window for the induction of fibrillation. Also provided are methods for delivering a stimulus to the heart of a subject during the determined vulnerable window.

The subject methods for determining a vulnerable window will be described first, followed by a description of methods for delivering a stimulus to the heart of a subject during the determined vulnerable window.

Methods for Determining an Optimal Vulnerable Window

As summarized above, the subject disclosure provides a method for delivering a stimulus to the heart of a subject, for instance, during a vulnerable window. In certain embodiments, a “vulnerable window” is the vulnerable period of the cardiac cycle during repolarization wherein cardiac fibrillation may be induced by the delivery of an electrical current. Accordingly, the subject methods are suitable for use with any device that is capable of delivering an electrical stimulus to the heart. Suitable devices for delivering an electrical stimulus to the heart are commonly known as defibrillators and/or defibrillation systems. Defibrillators may be external or internal (e.g., implantable).

Any well known defibrillator and/or defibrillator system may be used in accordance with the methods of the disclosure. For instance, in certain instances, a suitable defibrillator for use in delivering an electrical stimulus to the heart is an implantable cardiac defibrillator, such as those available from Medtronic, Inc. and St. Jude Medical, Inc. For example, the ICDs marketed by St. Jude Medical as the Photon®, Epic™, Epic™+ and Contour® families. Other examples of ICDs are disclosed in published U.S. patent application Ser. No. 10/437,110 (U.S. publication no. 20040002738), the entire disclosure of which is herein incorporated by reference.

In certain instances of the subject disclsoure, the vulnerable window is determined by obtaining a waveform, such as an intracardiac waveform, from a subject's heart; measuring an interval between a plurality of time points on the waveform, potentially including the pacing stimulus; and evaluating the morphology of the waveform and thereby determining the vulnerable window, for instance, for the induction of fibrillation.

A wave form, such as an intracardiac waveform, to be analyzed may be obtained by any suitable method well known in the art. For instance, a plurality of electrodes, e.g., leads, may be positioned at different positions on a subject's body and the electrical conductance between the electrodes can be measured so as to generate an intracardiac waveform. Specifically, in certain embodiments, one or two electrodes may be positioned so as to be in contact with the heart and/or another region of the body (e.g., another position on the heart) and the electrical conductance between the electrodes may be measured so as to produce a intracardiac waveform. The waveform, and hence the optimum window on the waveform, may change based on such variables as inter-electrode distance.

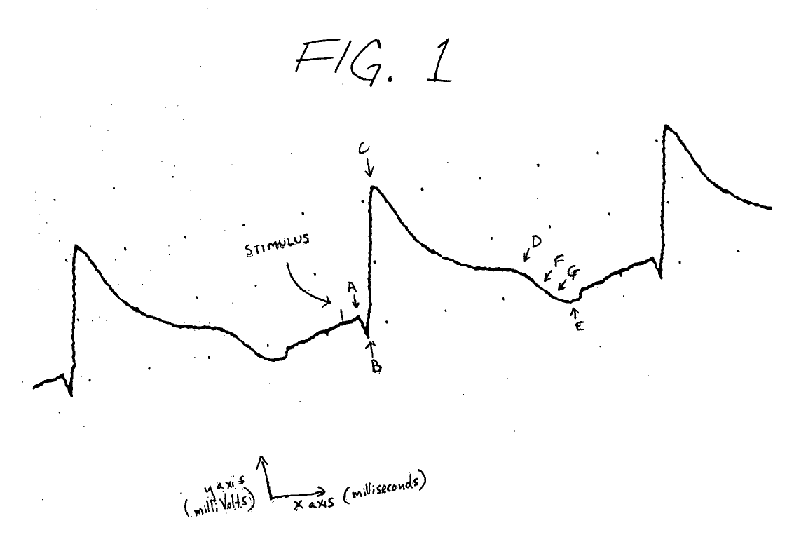

An exemplary intracardiac waveform obtained in accordance with the disclosed methods is illustrated in FIG. 1, although one of ordinary skill in the art will recognize that there are many variations of a intracardiac waveform and the waveform set forth in FIG. 1 is for illustrative purposes, and is not meant to limit the scope of the disclosure. As shown, the illustrated intracardiac waveform is graphically plotted, where the x-axis comprises time, typically measured in milliseconds, and the y-axis comprises voltage, typically measured in millivolts (for example).

Once a waveform, such as an intracardiac waveform, is obtained an interval between one or more time points, e.g., a plurality of time points, on the intracardiac waveform may be measured. The interval between the plurality of time points on the intracardiac waveform may be measured by any suitable measuring means and by any suitable methodology such as those well known in the art. For instance, the interval may be measured as a function of time and/or a function of distance, or the like. In certain embodiments, the waveform, e.g., intracardiac waveform, may be graphically plotted and the distance between the plurality of points may be measured, for instance, with a caliper. The plurality of time points, between which the interval is to be measured may be any two or more fixed time points on the waveform, potentially including a pacing stimulus.

For example, in one instance, once the intracardiac waveform(s) is obtained, the morphology of the waveform may be measured and the corresponding information/data may be recorded and/or analyzed so as to determine one or more characteristics of the waveform, e.g., intracardiac waveform. Specifically, by analyzing and/or evaluating the morphology of the waveform the optimal window of vulnerability may be determined. By the optimal window of vulnerability it is meant that point on the waveform whereat an electrical stimulus may be delivered to the heart so as to easily, e.g., most easily, induce fibrillation. With ULV testing, the point may correlate with the ability to induce fibrillation with the maximum voltage or energy.

This can be seen with reference to FIG. 1. As set forth above, FIG. 1, illustrates a representative cardiac waveform that may be obtained from a subject, e.g., by well known methods in the art, in accordance with the methods of the disclosure. The interval between any two time points may be measured, however, representative time points have been set out in FIG. 1 as time points A, B, C, D, F, G, and E. (See also, Table I, below). One or more of these representative time points may correlate with one or more well known characteristics of a cardiograph, e.g., an intracardiac electrogram, such as a trough, a peak, an inflection point, an onset of a stimulus, or the like.

Accordingly, as illustrated in FIG. 1, an interval to be measured may include an initial time point such as the onset of a pacing pulse or A, B, C, D, F, G, or E, or any point there between; and a later (e.g., a second) time point, which may include a time point after the initial time point, such as A, B, C, D, F, G, E, or any point therein between. For instance, the initial time point may be a time point such as a point on the intracardiac wave that indicates the onset of a pacing pulse or, alternatively, points A, B, or C; and the second time point may, therefore, be a later time point, such as D, F, G, E, or some point there between but after points A, B, or C. In such a situation, the interval will be the period between the onset of a pacing pulse or points A, B, or C and points D, F, G, E or any point there between. Hence, either the initial or later time points may include a peak, a trough, a return to baseline or zero crossing, and a point between a peak and a trough of the waveform.

Accordingly, in certain instances of the invention, the measurements of timing points, e.g., the interval, of the intracardiac waveform may include such timing points as set forth here below in Table I:

TABLE I

| Point/Item | Description |
|---|---|
| A | e.g., an initial start of deflection |
| B | e.g., a first trough |
| C | e.g., a peak |
| D | e.g., a point in the slope between point C and point E. |
| E | e.g., a second trough |
| F | e.g., a point in the slope between point D and point E |
| G | e.g., a point in the slope between point D and point E, for |
| | example 2/3 the distance between point D and E |
| H | e.g., a point in the slope between point F and point E |
| I | time period (msec) between stimulation to point A |
| J | time period (msec) between stimulation to point B |
| K | time period (msec) between stimulation to point C |
| L | time period (msec) between stimulation to point D |
| M | time period (msec) between stimulation to point E |
| N | time period (msec) between stimulation to point F |
| O | time period (msec) between stimulation to point G |
| P | time period (msec) between points D and E |
| Q | time period (msec) between points D and G |
| R | time period (msec) between points F and E |
| S | time period (msec) between points C and E |
| T | time period (msec) between points C and G |
| U | time period (msec) between points B and E |
| W | Time period (msec) between point A and any other point |
| | noted. |
| X | An inflection point |
| Y | Time period (msec) between any point noted and G |

In certain instances, a intracardiac waveform may be a waveform generated from a heart beat without previously being stimulated (e.g., the waveform may be the result of a native condition). Accordingly, the initial time point may include any one of A, B, C, or any point there between and the later (e.g., second) time point may include any of points D, F, G or E.

In other embodiments, a cardiac waveform may be a waveform generated from a heart beat generated as a result of an applied electrical stimulus. Such a stimulus may be a pacing stimulus/shock, a defibrillation shockwave (DSW), or the like, which may be applied via a defibrillator, such as an ICD. An intracardiac waveform generated as the result of the application of such a stimulus may be a repeatable, evoked, and/or entrainable response.

For example, in one instance, a stimuli and/or pacing shock is applied to the heart of a subject. The resulting intracardiac waveform(s) is then obtained and the interval is measured, wherein the interval includes a first time point, which may be a point on the intracardiac waveform at which pacing begins, and a second time point which may include a point on the intracardiac waveform after the beginning of the pacing. Accordingly, in certain instances, the method includes the delivery of a pacing stimulus to the heart of a subject, e.g., for pacing the heart. The pacing stimulus may be delivered by any means, such as means well known in the heart, for instance, such as via a defibrillator, e.g., an ICD.

The pacing may be at a set or variable rate. In certain instances, the pacing involves delivering a pulse of electric current to a subject's heart at a rate from about 60 bpm to about 220 bpm., such as a rate from about 100 bpm to about 160 bpm, including a rate from about 120 bpm to about 140 bpm. In certain instances, the pacing continues for a set duration. For instance, the pacing duration may be for a duration from about 2 to about 220 beats, such as a duration from about 5 to about 100 beats, including a duration from about 8 to about 40 beats.

Accordingly, where the initial time point is demarcated by the onset of a pacing shock/stimulus, as indicated with reference to FIG. 1, the second time point may include any of points A, B, C, D, F, G or E, or any point there in between.

However, in certain instances, the second time point includes D, F, G or E, or any point there in between.

Hence, in certain instances, the later or second time point may be point D or may be a point determined by a mathematical relationship between point D and another point on the waveform, such as a mathematical relationship of a fraction of the distance between points D and E. In certain instances, the second point may be point E or may be a point determined by a mathematical relationship between point E and another point on the waveform, including, for example, point F as an inflection point. For instance, in certain instances, the second point as point G may be located about half of the distance between point F as an inflection point and point E. In certain instances, the later or second time point may be point G or may be a point determined by a mathematical relationship between point E and another point on the waveform. For instance, in certain instances, and as illustrated in FIG. 1, point G may be located about ⅔ of the distance between points D and E. In certain instances, the second point may be point F or may be a point determined by a mathematical relationship between point F and another point on the waveform. For instance, the mathematical relationship may be determined by a mathematical function such as a Fourier transformation or a Bartlett transformation; or a relationship between shockwave characteristics, a wavelet analysis, or a combination thereof.

Accordingly, once the resulting intracardiac waveform(s) is obtained, the morphology of the cardiac wave is measured and/or the corresponding information/data is recorded and/or analyzed so as to determine one or more characteristics of the intracardiac waveform. Specifically, by analyzing and/or evaluating the morphology of the waveform, e.g., the time interval between two points on the cardiac wave, the “sweet spot” of the window of vulnerability may be determined. For instance, in certain embodiments, the vulnerable window may include points D, F, G, E, or a point there between on the intracardiac waveform, such as a point that is approximately ⅔ distance between points D and E, or point G.

By optimal window of vulnerability is meant that point on the intracardiac waveform whereat an electrical stimulus may be delivered to the heart so as to induce fibrillation.

Hence, the optimal point of the window of vulnerability may be determined so as to verify the efficiency of a defibrillator (e.g., a implantable cardiac defibrillator) or defibrillator system, for instance, during defibrillation threshold testing, such as upper limit of vulnerability (ULV) or non-ULV based testing. Therefore, in one aspect of the subject disclosure, the time period between points or portions of the intracardiac waveform are measured and/or analyzed to determine the optimal point of the window of vulnerability, and once determined a stimuli or shockwave is delivered to the heart during the vulnerable window so as to induce fibrillation.

### EXAMPLES

The following is set forth so as to provide those skilled in the art with a complete disclosure and description of how to make and use embodiments in accordance with the invention, and is not intended to limit the scope of what the inventor regards as the invention. Efforts have been made to ensure accuracy with respect to amounts used (e.g. the amount of milliseconds, millivolts, etc.) but some experimental errors and deviations should be accounted for.

Experiments may be run in accordance with the methods of the subject disclosure. Although cardiac waveforms will differ, for example with changes in intra-electrode distance and with individual patient variations, the same general waveform and protocol may be employed. Accordingly, the following is the general protocol that may be practiced and the results that may be obtained.

In a method for determining the vulnerable window for the induction of fibrillation, a St. Jude ICD model V-243 with a Riata lead may be used. The ICD may be used to pace the heart of a patient at a rate of about 120 beats per minute for approximately 8 to 120 beats. The intracardiac waveform may be measured with a Merlin programmer in the RV coil to RV tip configuration. The interval to be measured may be the interval between the pacing stimulus and the point corresponding to ⅔ of the distance between points D and E along the x axis (e.g., point G). Scanning is performed in 10, 20, 30 and 40 ms increments to either side of this interval if an accurate ULV or DFT are not determined. Alternatively, the interval to be measured may be the interval between the pacing stimulus and the point corresponding to ½ the distance between points F, (with F, for example, as an inflection point) and E along the x axis (e.g. point G). Scanning is performed in 10, 20, 30 and 40 ms increments to either side of this interval if an accurate ULV or DFT are not determined. Alternatively, point G may be determined based on a mathematical relationship between points F and E, or D and F. Additionally, waveform vectors may be used.

With respect to the Example, set forth above, and the claims, set forth below, it is noted although the shape of cardiac waveforms (e.g., intracaradiac waveforms) may differ, e.g., slightly, from one person to the next, the intracardiac waveform set forth in FIG. 1, is generally representative of all intracardiac waveforms, such as those obtained from the beating of a human heart.

Accordingly, although an actual intracardiac waveform for any given subject will be different, e.g., slightly different, to that set forth in FIG. 1, the waveform will typically have the same general shape as that set forth in FIG. 1.

Therefore, the waveform obtained from any given subject should be similar (e.g., should have a similar morphology) to the one set forth in FIG. 1, and will consequently, have time points A, B, C, D, G, E, F, as well as time points there between and the like, which are similar to time points A, B, C, D, G, E, F, as well as time points there between and the like, which are representative of typical time points and set out in FIG. 1.

Hence, what is meant by the term “similar” in reference to an actual time point or period (e.g., A, B, C, D, G, E, F, or the like) on a intracardiac waveform actually obtained from a subject is the time point or period (e.g., A, B, C, D, G, E, F, or the like) that corresponds to the representative time point or period (e.g., A, B, C, D, G, E, F, or the like) as set forth in FIG. 1. These time points (e.g., A, B, C, D, G, E, F, or the like) in the intracardiac waveform actually obtained from the subject can easily be determined by comparing the actual waveform obtained from the subject to the general waveform set forth in FIG. 1 and demarcating time points (e.g., A, B, C, D, G, E, F, or the like) on the actual obtained waveform that correspond to time points (e.g., A, B, C, D, G, E, F, or the like) set forth in FIG. 1. Additionally, as described herein, the actual time points corresponding to the representative time points (e.g., A, B, C, D, G, E, F, or the like) in FIG. 1, can be precisely determined using mathematical formulae well known in the art, such as a Fourier transformation, a Bartlett transformation, a relationship between characteristics, and a wavelet analysis, or a combination thereof. In this manner, time points and/or periods (A, B, C, D, G, E, F, as well as time points there between and the like) as set forth in FIG. 1 may be demarcated on an actual intracardiac wave form obtained from a subject and the time points and/or periods may be measured and relationships there between derived as disclosed herein above. In certain instances, the morphology of the cardiac waveform obtained from the subject will be identical to the morphology of the representative waveform set forth in FIG. 1, such that time points and/or periods (A, B, C, D, G, E, F, as well as time points there between and the like) may be directly transposed from FIG. 1 to the cardiac waveform obtained from the subject.

All publications and patents cited in this specification are herein incorporated by reference as if each individual publication or patent were specifically and individually indicated to be incorporated by reference.

While the invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the invention. All such modifications are intended to be within the scope of the claims appended hereto.

What is claimed is:

1. A method of determining an optimal vulnerable window for the induction of fibrillation in a subject, the method comprising
(a) obtaining an intracardiac waveform from the subject's heart;
(b) measuring an interval between a plurality of time points on said intracardiac waveform; and
(c) evaluating the morphology of the waveform in such a manner as to determine the vulnerable window for the induction of fibrillation.

2. The method according to claim 1, wherein the intracardiac waveform is produced by native conduction or as an evoked response.

3. The method according to claim 1, wherein said intracardiac waveform is obtained from at least two electrodes.

4. The method according to claim 1, wherein at least one of said electrodes comprise a lead that is in contact with said subject's heart.

5. The method according to claim 1, wherein said interval comprises the difference between two fixed points on said intracardiac waveform.

6. The method according to claim 1, further comprising pacing the heart.

7. The method according to claim 6, wherein said pacing is at a set rate.

8. The method according to claim 7, wherein said pacing comprises delivering a pulse of electric current to said subject's heart at a rate from about 60 bpm to about 220 bpm.

9. The method according to claim 8, wherein said pacing comprises delivering a pulse of electric current to said subject's heart at a rate from about 100 bpm to about 160 bpm.

10. The method according to claim 8, wherein said pacing comprises delivering a pulse of electric current to said subject's heart at a rate from about 120 bpm to about 140 bpm.

11. The method according to claim 6, wherein said pacing continues for a set duration.

12. The method according to claim 11, wherein said set duration comprises from about 2 to about 220 beats.

13. The method according to claim 12, wherein said set duration comprises from about 5 to about 100 beats.

14. The method according to claim 13, wherein said set duration comprises from about 8 to about 40 beats.

15. The method according to claim 6, wherein said interval between said plurality of time points comprises a first time point comprising a point on said intracardiac waveform at which said pacing begins and a second time point comprising a point on said intracardiac waveform after the beginning of said pacing.

16. The method according to claim 15, wherein said second point includes a peak, a trough, an inflection point, a return to baseline or zero crossing, and a point between a peak and a trough of the waveform, or a mathematical relationship between these noted points.

17. The method according to claim 15, wherein said intracardiac waveform comprises a morphology similar to that of FIG. 1.

18. The method according to claim 17, wherein said second time point on said intracardiac waveform comprises a time point selected from the group consisting of time points on the intracardiac waveform that are similar to A, B, C, D, F, G, and E of FIG. 1.

19. The method according to claim 18, wherein said second time point comprises a time point selected from the group consisting of time points similar to D, F, G, and E of FIG. 1.

20. The method according to claim 19, wherein said second point comprises a time point similar to point G of FIG. 1.

21. The method according to claim 20, wherein point G is about ⅔ of the distance between points D and E, or alteratively wherein point G is about ½ of the distance between points F and E, wherein F is an inflection point.

22. The method according to claim 19, wherein said second point comprises a time point similar to point D of FIG. 1.

23. The method according to claim 18, wherein said second point comprises a point determined by a mathematical relationship comprising time points similar to point D or F of FIG. 1 and another point on the waveform.

24. The method according to claim 23, wherein the mathematical relationship is determined by a mathematical function selected from the group consisting of: a Fourier transformation, a Bartlett transformation, a relationship between characteristics, and a wavelet analysis, or a combination thereof.

25. The method according to claim 23, wherein said second point comprises a point determined by a mathematical relationship of a fraction of the distance between points similar to D and E of FIG. 1, or alternatively wherein said second point compromises a point determined by a mathematical relationship of a fraction of the distance between points similar to points F and E of FIG. 1, wherein F may be an inflection point.

26. The method according to claim 18, wherein said second point comprises a time point similar to point E of FIG. 1.

27. The method according to claim 18, wherein said second point comprises a point determined by a mathematical relationship comprising a time point similar to point E of FIG. 1 and another point on the waveform.

28. The method according to claim 27, wherein the mathematical relationship is determined by a mathematical function selected from the group consisting of: a Fourier transformation, a Bartlett transformation, a relationship between characteristics, and a wavelet analysis, or a combination thereof.

29. The method according to claim 18, wherein said second point comprises a time point similar to point F of FIG. 1.

30. The method according to claim 18, wherein said second point comprises a point determined by a mathematical relationship comprising a time point similar to point F of FIG. 1 and another point on the waveform.

31. The method according to claim 30, wherein the mathematical relationship is determined by a mathematical function selected from the group consisting of: a Fourier transformation, a Bartlett transformation, a relationship between characteristics, and a wavelet analysis, or a combination thereof.

32. The method according to claim 1, wherein said intracardiac waveform comprises a wave form similar to FIG. 1 and said interval between said plurality of time points comprises a first time point comprising a time point selected from the group consisting of a pacing stimulus, a point similar to A, B, C, D, F, G, and E of FIG. 1, and a second time point comprising a later point in time on said intracardiac waveform.

33. The method according to claim 32, wherein said first time point comprises a point on said intracardiac waveform selected from the group consisting of a pacing stimulus, or a point similar to points A, B, or C of FIG. 1.

34. The method according to claim 33, wherein said second point includes a peak, a trough, a return to baseline or zero crossing, and a point between a peak and a trough of the waveform.

35. The method according to claim 33, wherein said second time point comprises a time point similar to the time points selected from the group consisting of D, F, G, and E of FIG. 1.

36. The method according to claim 1, wherein said vulnerable window is determined during defibrillation threshold testing.

37. The method according to claim 36, wherein said defibrillation threshold testing comprises upper limit of vulnerability (ULV) or non-ULV based testing.

38. A method for delivering a stimuli to the heart of a subject so as to induce fibrillation, comprising
(a) determining a vulnerable window for the induction of fibrillation in accordance with the method according to claim 1; and
(b) delivering said stimuli to said heart during said vulnerable window so as to induce fibrillation.

39. The method according to claim 38, wherein said intracardiac waveform comprises a waveform similar to that of FIG. 1 and said vulnerable window comprises a time point similar to point D, F, G, E of FIG. 1, or a point there between on said intracardiac waveform.

40. The method according to claim 39, wherein said vulnerable window comprises a point on the intracardiac waveform that is approximately ⅔ distance between similar points to points D and E of FIG. 1, or alternatively comprises a point that is approximately ½ the distance between similar points to points F and E of FIG. 1, where point F may be an inflection point.

41. The method according to claim 39, wherein said vulnerable window comprises a time point similar to point G of FIG. 1.

42. The method according to claim 38, wherein said stimuli is delivered so as to verify the efficacy of a defibrillator system.

43. The method according to claim 38, wherein said defibrillator system comprises an implantable cardiac defibrillator (ICD).
